(19) United States
(12) Patent Application Publication
(10) Pub. No.: US 2018/0103620 A1
(43) Pub. Date: Apr. 19, 2018

(54) METHODS AND MATERIALS FOR USING TRANSGENIC NON-HUMAN ANIMALS EXPRESSING SODIUM IODIDE SYMPORTER POLYPEPTIDES TO MONITOR PATHOLOGICAL PROCESSES, DISEASE PROGRESSION, OR THERAPEUTIC RESPONSES OVER TIME

(71) Applicant: Mayo Foundation for Medical Education and Research, Rochester, MN (US)

(72) Inventors: Kah-Whye Peng, Rochester, MN (US); Stephen James Russell, Rochester, MN (US); Patrycja Lech, Oronoco, MN (US); Lukkana Suksanpaisan, Rochester, MN (US)

(73) Assignee: Mayo Foundation for Medical Education and Research, Rochester, MN (US)

(21) Appl. No.: 15/566,827

(22) PCT Filed: Apr. 15, 2016
(86) PCT No.: PCT/US16/27876

§ 371 Date: Oct. 16, 2017

Related U.S. Application Data
(60) Provisional application No. 62/149,425, filed on Apr. 17, 2015.

(51) Int. Cl. A01K 67/027; A61K 51/02

(52) U.S. Cl. CPC A01K 67/0275; A61K 51/025; A01K 2207/20; C12N 2015/859; A01K 2267/0393; A01K 2217/05; A01K 2227/105

(57) ABSTRACT

This document provides methods and materials for using live transgenic non-human animals (e.g., non-human mammals) to perform radiotracer imaging to monitor pathological processes, disease progression, or therapeutic responses in vivo over time. For example, methods and materials for using live transgenic non-human animals (e.g., non-human mammals) having somatic and germline cells that include nucleic acid (e.g., a transgene) having a promoter sequence operably linked to a nucleic acid sequence encoding a NIS polypeptide to perform radiotracer imaging to monitor pathological processes, disease progression, or therapeutic responses in vivo over time are provided.

### CROSS-REFERENCE TO RELATED APPLICATIONS

This application claims benefit of the U.S. Provisional Application Ser. No. 62/149,425, filed on Apr. 17, 2015. The disclosure of the prior application is considered part of the disclosure of this application, and is incorporated in its entirety into this application.

### 1. TECHNICAL FIELD

This document relates to methods and materials involved in radiotracer imaging using live transgenic non-human animals (e.g., non-human mammals) to monitor pathological processes, disease progression, or therapeutic responses in vivo over time. For example, this document relates to using live transgenic non-human animals (e.g., non-human mammals) having somatic and germline cells that include nucleic acid (e.g., a transgene) having a promoter sequence operably linked to a nucleic acid sequence encoding a sodium iodide symporter (NIS) polypeptide to monitor pathological processes, disease progression, or therapeutic responses in vivo over time.

### 2. BACKGROUND INFORMATION

NIS polypeptides mediate the uptake and concentration of iodide in the thyroid gland, providing the basis for diagnostic thyroid radioimaging and radioiodine therapy. When nucleic acid encoding a NIS polypeptide is introduced into non-thyroid cells, they can, like thyroid cells, be detected using iodide/pertechnetate radioimaging and/or destroyed using iodide radiotherapy.

### SUMMARY

This document provides methods and materials for using live transgenic non-human animals (e.g., non-human mammals) to perform radiotracer imaging to monitor pathological processes, disease progression, or therapeutic responses in vivo over time. For example, this document provides methods and materials for using live transgenic non-human animals (e.g., non-human mammals) having somatic and germline cells that include nucleic acid (e.g., a transgene) having a promoter sequence operably linked to a nucleic acid sequence encoding a NIS polypeptide to perform radiotracer imaging to monitor pathological processes, disease progression, or therapeutic responses in vivo over time. In some cases, transgenic NIS non-human animals (e.g., mice or larger mammals) can be used as described herein for non-invasive quantitation and localization of changing levels of NIS polypeptide expression through the various stages of a pathological process of interest in a single living animal.

NIS radiotracers can be efficiently concentrated by cells expressing a NIS polypeptide, and SPECT, PET, and γ-camera images can be used to monitor the in vivo biodistribution of NIS radiotracers. As described herein, imaging techniques that involve the uptake of radiotracers such as ¹²³I, ¹²⁴I, ¹²⁵I, B¹⁸F₄ (tetrafluoroborate), and ^{99m}TcO₄ (pertechnetate) by expressed NIS polypeptides of a transgenic non-human animal (e.g., non-human mammal) provided herein can be used for accurate, sensitive, high resolution detection of NIS polypeptide expression and monitoring of pathological processes, disease progression, or therapeutic responses in vivo over time in living mammals (e.g., for longitudinal studies).

Unlike luciferase and GFP reporter genes, which rely on optical imaging, NIS polypeptides can be used for quantitative expression monitoring in large mammals as well as in deep-seated tissues (e.g., mouse tissues) because, unlike photons, gamma rays are minimally attenuated by the tissues through which they pass.

The methods and materials provided herein are based, at least in part, on the discovery that the sensitivity of detection of NIS polypeptide expressing cells in the context of a transgenic NIS animal is not too low to allow for the localization and quantitation of a pathological or developmental process within the transgenic animal even though other work may have indicated that NIS expressing cells could not be detected below a threshold number of at least about 100,000 cells in a single location (FIG. 15). The methods and materials provided herein also are based, at least in part, on the discovery that the changes in NIS polypeptide expression at sites of disease pathology are not too small to allow for accurate quantitative analyses over time. In addition, the methods and materials provided herein are based, at least in part, on the discovery that over-expression of a NIS polypeptide from a transgene at sites where it is not naturally expressed does not result in abnormal tissue development or other toxicities leading to premature death of the transgenic animal. Further, the methods and materials provided herein are based, at least in part, on the discovery that, while no regulatory element (e.g., promoter or enhancer) has a level of pathology specificity that does not result in at least some expression in normal tissues, this background NIS polypeptide expression in normal tissues does not confound the study of the pathological process itself.

As described herein, pathological processes, disease progression, and therapeutic responses can be assessed in vivo over time using the live transgenic non-human animals (e.g., non-human mammals) provided herein.

In general, one aspect of this document features a method of monitoring a pathological process over time within a living transgenic non-human animal comprising somatic and germline cells comprising a promoter sequence operably linked to a nucleic acid sequence encoding a NIS polypeptide. The method comprises, or consists essentially of, (a) administering a first preparation comprising a radiotracer to the animal during a first time point, wherein the radiotracer is transported into a cell that expresses the NIS polypeptide, (b) collecting imaging data of the radiotracer from within the animal to identify cells containing the radiotracer following the administration of the first preparation, (c) administering a second preparation comprising a radiotracer to the animal during a second time point after the first time point, and (d) collecting imaging data of the radiotracer from within the animal to identify cells containing the radiotracer following the administration of the second preparation. The pathological process can be fibrosis. The animal can be a mouse or rat. The promoter sequence can be a Col1α1 promoter sequence. The NIS polypeptide can be a human NIS polypeptide. The second time point can be at least 5 days after the first time point. The second time point can be at least 20 days after the first time point. The radiotracer can be pertechnetate or radioiodide.

In another aspect, this document features a transgenic non-human animal, whose somatic and germline cells comprise a transgene comprising a promoter sequence operably linked to a nucleic acid sequence encoding a NIS polypeptide, wherein radiotracer images of the animal at different time points, when a radiotracer that is transported into cells that express the NIS polypeptide is administered to the animal, provide imaging data with observable differences in the stages of a pathological process within the animal at the different time points. The pathological process can be fibrosis. The animal can be a mouse or rat. The promoter sequence can be a Col1α1 promoter sequence. The NIS polypeptide can be a human NIS polypeptide. The different time points can be at least 5 days apart. The different time points can be at least 20 days apart. The radiotracer can be pertechnetate or radioiodide.

In another aspect, this document features a transgenic non-human animal, whose somatic and germline cells comprise a transgene comprising a promoter sequence operably linked to a nucleic acid sequence encoding a NIS polypeptide, wherein the promoter sequence is a Col1α1 promoter sequence, an osteocalcin promoter sequence, a bone sialoprotein (BSP) promoter sequence, a VEGFR2 promoter sequence, a prostate-specific antigen (PSA) promoter sequence, an Epo promoter sequence, a serum amyloid A-1 (Saa1) promoter sequence, a TNF promoter sequence, an iNOS promoter sequence, a 3 NFkB site from the Igk light chain promoter sequence, an heme oxygenase-1 (Hol or Hmox-1) promoter sequence, a superoxide dismutase (SOD1) promoter sequence, a cytochrome p450 isoform 3A (Cyp3A4) promoter sequence, a rat insulin gene (RIP) promoter sequence, a resistin (Retn) promoter sequence, or an elastase I (EL1) promoter sequence.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram of a transgene construct used to make mice and rats transgenic for human NIS nucleic acid operably linked to a rat collagen α1(1) promoter.

FIG. 2 is a diagram of a transgene construct used to make mice and rats transgenic for human NIS nucleic acid operably linked to a human β-actin promoter.

FIG. 3 is a photograph of a gel showing the indicated PCR results.

FIG. 4. Plasmid construct used for derivation of transgenic mice and rats.

FIG. 4A. Plasmid map. FIG. 4B. Radioiodine uptake assay in transfected HT1080 cells. FIG. 4C. MluI/PvuI digest. The 7.6 kb fragment was used for pronuclear injections.

FIG. 5 contains photographs of SPECT-CT images obtained from a control non-transgenic rat injected with Tc99m (1 mCi/animal).

FIG. 6 contains photographs of SPECT-CT images obtained from a transgenic rCol1α1pro/hNIS rat injected with Tc99m (1 mCi/animal).

FIG. 7 contains photographs of SPECT-CT images obtained from a transgenic hActinpro/hNIS rat injected with Tc99m (1 mCi/animal). High NIS expression was observed in heart muscle. Even with this ubiquitous NIS expression, the rats were healthy and fertile.

FIG. 8 contains photographs of SPECT-CT images obtained from three transgenic rCol1α1pro/hNIS mice and one control non-transgenic mouse injected with Tc99m (1 mCi/animal) and monitored over the indicated times.

FIG. 9. SPECT-CT image obtained from transgenic rCol1α1pro/hNIS mouse demonstrating NIS signal in the bone.

FIG. 10 contains photographs of SPECT-CT images obtained from three transgenic rCol1α1pro/hNIS rats with induced rotator cuff injury to the left shoulder. The rats were injected with Tc99m (1 mCi/animal) and monitored over the indicated times.

FIG. 11 contains photographs of SPECT/CT images obtained from transgenic rCol1α1pro/hNIS rat with induced rotator cuff injury to the left shoulder demonstrating change in NIS expression over time (day 3 and day 35) during healing process.

FIG. 12 contains photographs of SPECT-CT images obtained from transgenic rCol1α1pro/hNIS rats and control non-transgenic rats with induced rotator cuff injury to the left shoulder. The rats were injected with Tc99m (1 mCi/animal) and monitored over the indicated times.

FIG. 13. One hour before SPECT-CT imaging, rats were injected with Tc99m at 1 mCi/rat. NIS signals on both shoulders were quantitated using PMOD program. Graph shows the fold increase of NIS uptake signal on the injured over control shoulder in each animal. Rat #20.3.37 is PCR negative and was used as negative control. There is no increase in NIS expression between both shoulders in the control rat.

FIG. 14 contains photographs of SPECT-CT images obtained from five transgenic rCol1α1pro/hNIS rats after rotator cuff injury was induced to the left shoulder. The rats were injected with Tc99m (1 mCi/animal).

FIG. 15 demonstrates the sensitivity of detection of NIS expressing cells by SPECT/CT. 2e5, 6.7e5, and 2e6 dog MSC expressing NIS were transplanted subcutaneously into mice (A), and the animals were imaged by SPECT/CT (B).

### DETAILED DESCRIPTION

This document provides methods and materials for using live transgenic non-human animals (e.g., non-human mammals) to perform radiotracer imaging to monitor pathological processes, disease progression, or therapeutic responses in vivo over time. Examples of pathological processes that can be monitored in vivo over time using the methods and materials provided herein include, without limitation, wound healing, organ fibrosis, tumorigenesis, growth and dissemination of spontaneously occurring tumors, and inflammation. In some cases, the methods and materials provided herein can be used to monitor immune responses in vivo over time.

As described herein, a transgenic non-human animal (e.g., transgenic non-human mammal) can be designed to have somatic and germline cells that include nucleic acid (e.g., a transgene) having a promoter sequence operably linked to a nucleic acid sequence encoding a NIS polypeptide. Such transgenic non-human animals can be farm animals such as pigs, goats, sheep, cows, and horses, rabbits, rodents such as rats, guinea pigs, and mice, and non-human primates such as baboons, monkeys, and chimpanzees. The term “transgenic non-human animal” as used herein includes, without limitation, founder transgenic non-human animals as well as progeny of the founders, progeny of the progeny, and so forth, provided that the progeny retain the transgene.

The nucleated cells of the transgenic non-human animals provided herein can contain a transgene that includes a promoter sequence (e.g., a collagen lα(1) promoter sequence) operably linked to a nucleic acid sequence encoding a NIS polypeptide. In general, the promoter sequence and nucleic acid sequence encoding a NIS polypeptide of a transgene described herein are heterologous. A promoter sequence of a transgene described herein can be one that drives NIS polypeptide expression in cells involved in a particular pathological process, cells involved in progression of a particular disease, cells involved in a particular immune response, or cells involved in a particular therapeutic response. For example, a transgene can be designed to make a transgenic animal (e.g., a transgenic mammal) where a collagen lα(1) promoter sequence drives NIS polypeptide expression. Such a transgenic animal can be used to assess organ fibrosis. Other examples of promotors that can be used to drive NIS polypeptide expression within a transgenic animal to monitor a particular condition (e.g., a pathological process or disease progression) are set forth in Table 1.

TABLE 1

| Promoter Sequence for driving NIS | Condition to be monitored |
|---|---|
| polypeptide expression | in transgenic animal |
| Osteocalcin promoter (hOC) | Bone repair/osteopathy |
| Bone sialoprotein (BSP) promoter | Matrix deposition/osteopathy |
| VEGFR2 gene promoter (VEGF) | angiogenesis |
| PSA promoter | inflammation |
| Epo promoter | inflammation |
| Serum amyloid A-1 promoter (Saa1) | inflammation |
| TNF promoter | inflammation |
| iNOS gene promoter | inflammation |
| 3 NFkB site from the Igk | inflammation |
| light chain promoter | |
| Heme oxygenase-1 promoter | Drug metabolism/toxicology |
| (Ho1 or Hmox-1) | and hypoxia |
| Superoxide dismutase promoter (SOD1) | Drug metabolism/toxicology |
| | toxicity |
| Cytochrome p450 isoform 3A promoter | Drug metabolism/toxicology |
| (Cyp3A4) | toxicity |
| Rat insulin gene promoter (RIP) | Atrophy |
| Resistin promoter (Retn) | Obesity |
| Elastase I promoter (EL1) | Neoplasia |
| Prostate-specific antigen promoter (PSA) | Neoplasia |

In some cases, a regulatory element other than a promoter sequence can be used to direct the expression of a NIS polypeptide of a transgene to particular cells. For example, a transgene can be designed to include a non-specific promoter in combination with a tissue- or cell-specific enhancer.

As described herein, a transgene used to make a transgenic non-human animal provided herein can include a nucleic acid sequence that encodes a NIS polypeptide. Any appropriate nucleic acid sequence that encodes a NIS polypeptide can be used as described herein. For example, a nucleic acid sequence that encodes a human, mouse, rat, monkey, dog, horse, cattle, or pig NIS polypeptide can be used. In some cases, a nucleic acid sequence that encodes a mutant NIS polypeptide can be used, provided that the mutant NIS polypeptide retains the ability to transport at least one radioactive tracer. Examples of radioactive tracers that can be transported into cells via a NIS polypeptide include, without limitation, ¹²³I, ¹²⁴I, ¹²⁵I, ¹³¹I, B¹⁸F₄^{, 186}ReO₄^{, 188}ReO₄, ^{99m}TcO₄, ⁹⁴TcO₄, and ³⁶ClO₄. For example, a nucleic acid sequence that encodes a wild-type or mutant NIS polypeptide that transports radioiodide and/or pertechnetate can be used included as part of a transgene of a transgenic non-human animal provided herein. An example of a mutant NIS polypeptide that can be used as described herein includes, without limitation, NIS-93E.

The term “operably linked” as used herein refers to positioning a regulatory element (e.g., a promoter sequence, an inducible element, or an enhancer sequence) relative to a nucleic acid sequence encoding a polypeptide (e.g., a NIS polypeptide) in such a way as to permit or facilitate expression of the encoded polypeptide. In the transgenes disclosed herein, for example, a promoter sequence (e.g., a Col1α1 promoter sequence) can be positioned 5′ relative to a nucleic acid encoding a NIS polypeptide (e.g., a human NIS polypeptide).

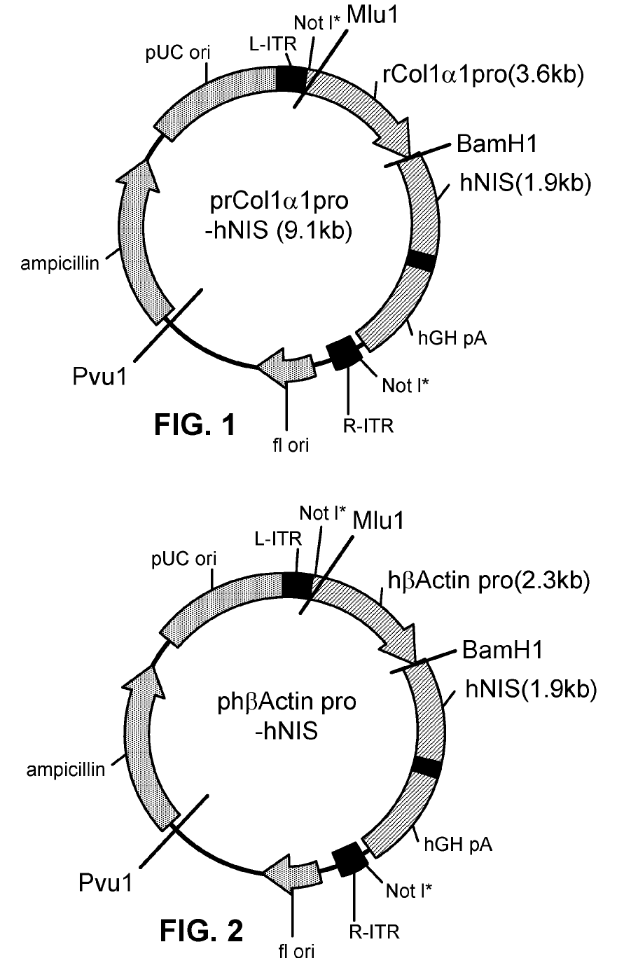

Various techniques can be used to introduce transgenes into non-human animals to produce founder lines, in which the transgene is integrated into the genome. Such techniques include, without limitation, pronuclear microinjection (See, e.g., U.S. Pat. No. 4,873,191), retrovirus mediated gene transfer into germ lines (Van der Putten et al., Proc. Natl. Acad. Sci. USA, 82:6148-1652 (1985)), gene targeting into embryonic stem cells (Thompson et al., Cell 56:313-321 (1989)), electroporation of embryos (Lo, Mol. Cell. Biol., 3:1803-1814 (1983)), and in vitro transformation of somatic cells, such as cumulus or mammary cells, followed by nuclear transplantation (Wilmut et al., Nature, 385:810-813 (1997); and Wakayama et al., Nature, 394:369-374 (1998)). For example, fetal fibroblasts can be genetically modified to contain a Col1α1pro/hNIS construct (FIG. 1), and then fused with enucleated oocytes. After activation of the oocytes, the eggs are cultured to the blastocyst stage. See, for example, Cibelli et al., Science, 280:1256-1258 (1998). Standard breeding techniques can be used to create animals that are homozygous for the transgene from the initial heterozygous founder animals. Homozygosity is not required, however, as the phenotype can be observed in hemizygotic animals. In some cases, a transgenic NIS non-human animal provided herein can be homozygous for the NIS transgene. In some cases, a transgenic NIS non-human animal provided herein can be heterozygous for the NIS transgene.

Once transgenic non-human animals have been generated, expression of an encoded NIS polypeptide can be assessed. Initial screening can be accomplished by Southern blot analysis to determine whether or not integration of the transgene has taken place. For a description of Southern analysis, see sections 9.37-9.52 of Sambrook et al., 1989, Molecular Cloning, A Laboratory Manual, second edition, Cold Spring Harbor Press, Plainview; NY. Polymerase chain reaction (PCR) techniques also can be used in the initial screening. PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length.

Expression of a nucleic acid sequence encoding a NIS polypeptide in particular cells of interest in a transgenic non-human animal can be assessed using techniques that include, without limitation, Northern blot analysis of tissue samples obtained from the animal, in situ hybridization analysis, Western analysis, immunoassays such as enzyme-linked immunosorbent assays, and reverse-transcriptase PCR (RT-PCR). In some cases, a radiotracer or combination of radiotracers can be given to the animal, and radiotracer imaging techniques can be used to identify those cells within the animal that express a NIS polypeptide. Control, non-transgenic animals can be used to differentiate those cells that express an endogenous NIS polypeptide from those cells that express a NIS polypeptide from a transgene.

Once a transgenic non-human animal having a nucleic acid (e.g., a transgene) that includes a regulatory element (e.g., a promoter) and a nucleic acid sequence that encodes a NIS polypeptide is obtained, a radiotracer or combination of radiotracers can be given to the transgenic animal, and radiotracer imaging techniques can be used to identify those cells within the transgenic animal that express the NIS polypeptide from the transgene. Again, control, non-transgenic animals can be used to differentiate those cells that express an endogenous NIS polypeptide from those cells that express a NIS polypeptide from a transgene. In some cases, the same transgenic non-human animal or group of transgenic non-human animals can be repeatedly administered a radiotracer or combination of radiotracers and imaged over time (e.g., days, weeks, months, or years) to monitor pathological processes, disease progression, or therapeutic responses within the living animals.

The invention will be further described in the following examples, which do not limit the scope of the invention described in the claims.

### EXAMPLES

### Example 1—Transgenic NIS Animals

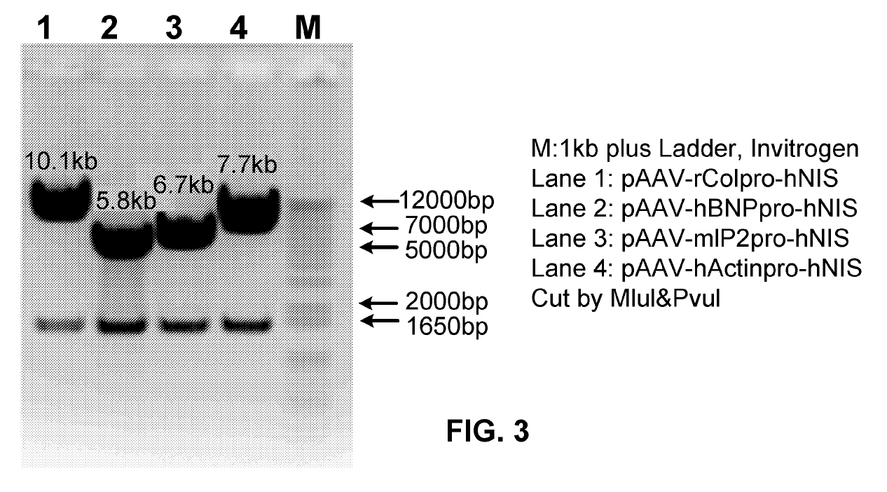

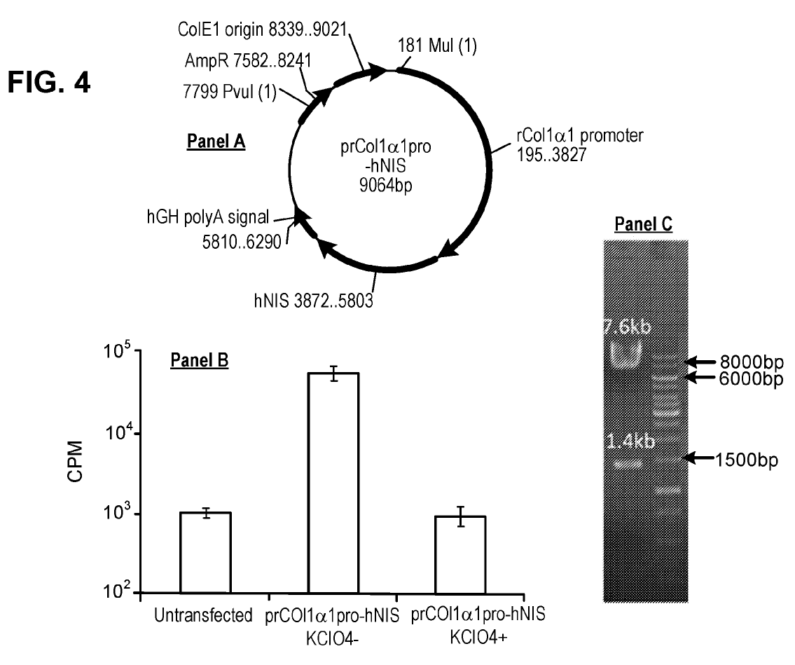

A human NIS (hNIS) cDNA was subcloned into an expression vector, and hNIS expression was driven by a 3.6 kb fragment of the rat collagen α1(1) promoter (rCol1α1pro; FIG. 1), a 2.3 kb fragment of the human beta actin promoter (hActinpro; FIG. 2), a human brain natriuretic peptide (BNP) promoter (hBNPpro), or a mouse insulin promoter (mIP2pro).

The DNA constructs were injected into zygotes and transferred into pseudopregnant female recipients, which resulted in the birth of offspring rats. Pups were imaged by SPECT/CT imaging and screened for hNIS expression. Tail snips were taken, and PCR analysis was performed to genotype the rodents (FIG. 3). Three pairs of primers with specificity for human NIS and not rat or mouse NIS were used to identify the transgenic animals. The primer pairs were set 1 (1489.1511: GCTCTCCTCC-CTGCTAACGACTC, SEQ ID NO:1; and 1835.1813: AGACGATCCTCATTG-GTGGGCAG, SEQ ID NO:2), set 2 (1447.1467: TGCGTGGCTCTCTCAGTCAAC, SEQ ID NO:3; and 1781.1761: AGTTCTTCAGGACCCTTGACC, SEQ ID NO:4), and set 3 (1447.1467: TGCGTGGCTCTCTCAGTCAAC, SEQ ID NO:5; and 1824.1805: ATTGGTGGGCAGGAAGCCAG, SEQ ID NO:6).

As confirmed from the PCR results, 6 female and 3 male transgenic rCol1α1pro/hNIS rats, 7 female and 12 male transgenic hActinpro/hNIS rats, 12 female and 6 male transgenic hBNPpro/hNIS rats, and 8 female and 7 male transgenic mIP2pro/hNIS rats were obtained. In addition, 3 female and 7 male transgenic rCol1α1pro/hNIS mice, 3 female and 9 male transgenic hActinpro/hNIS mice, 1 female and 5 male transgenic hBNPpro/hNIS mice, and 6 female and 7 male transgenic mIP2pro/hNIS mice were obtained.

The transgenic animals were quarantined for 8 weeks to insure that they did not have any transmissible parasites or diseases. While in quarantine, two transgenic rCol1α1pro/hNIS rats started to exhibit thin hair coats and gait abnormalities. These abnormalities were monitored on a weekly basis. Rat #579 (male) exhibited a uniformly thin fur coat, hind leg dysmetria (e.g., ataxia with lack of coordination of movement, hypermetria-ataxia with overreaching), and a hind limb proprioceptive deficit, and occasionally walked on curled toes. Rat #576 (female) exhibited a uniformly thin fur coat, kyphosis, and mild hind leg dysmetria (hypermetria). The rats remained stable since these abnormalities were first noted.

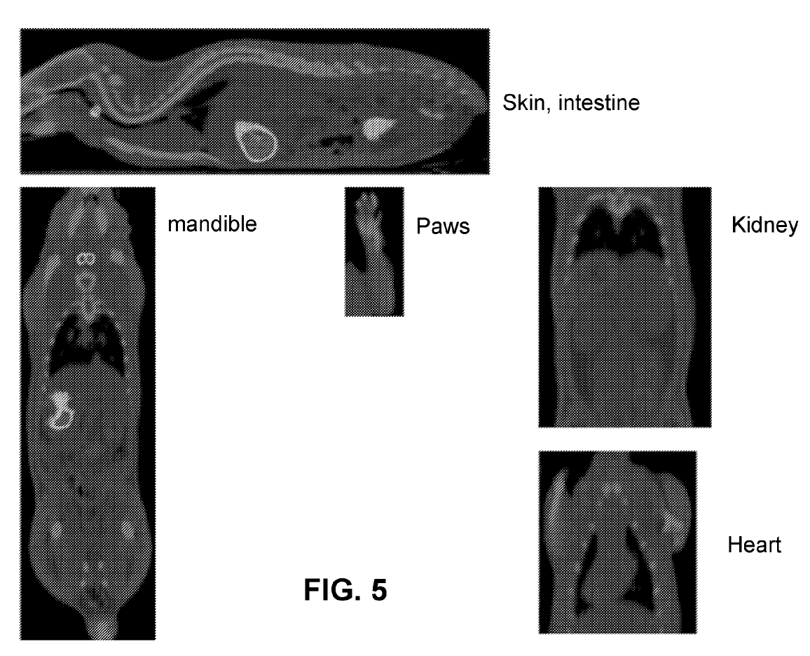

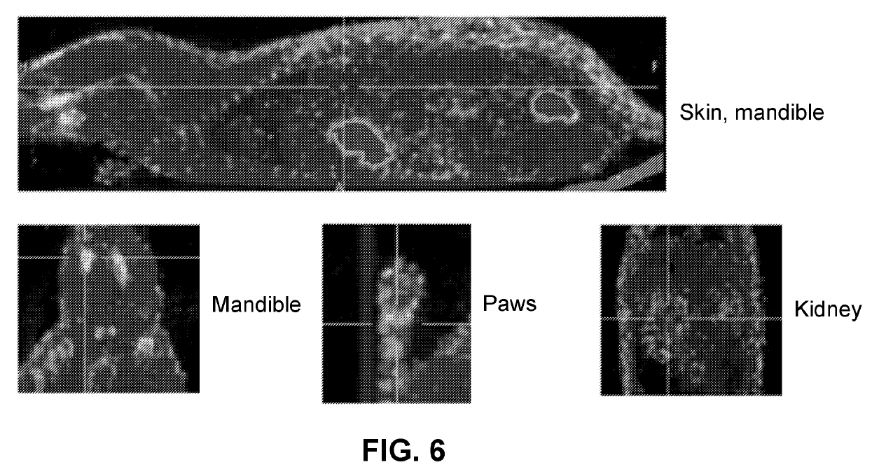

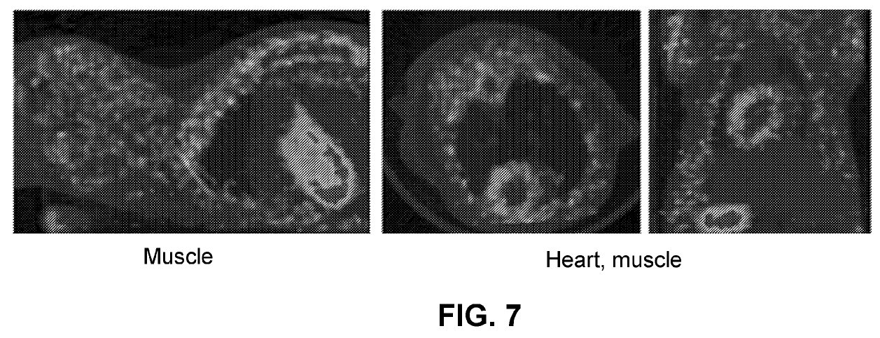

After all animals completed quarantine, SPECT/CT and I¹²⁵ or Tc99m pertechnetate imaging was performed. PCR and positive imaging animals were then paired with a wild-type mates to produce the F1 generation. Heterozygous littermate animals were then continuously bred until homogyzyous. DNA from a tail snip was used at the age of 3 weeks or older for PCR screening with two pairs of primers specific for the transgenic construct. PCR confirmed transgenic animals were screened for NIS expression using SPECT-CT. SPECT-CT imaging confirmed the expression of NIS above that observed in wild type control animals (FIGS. 5-7).

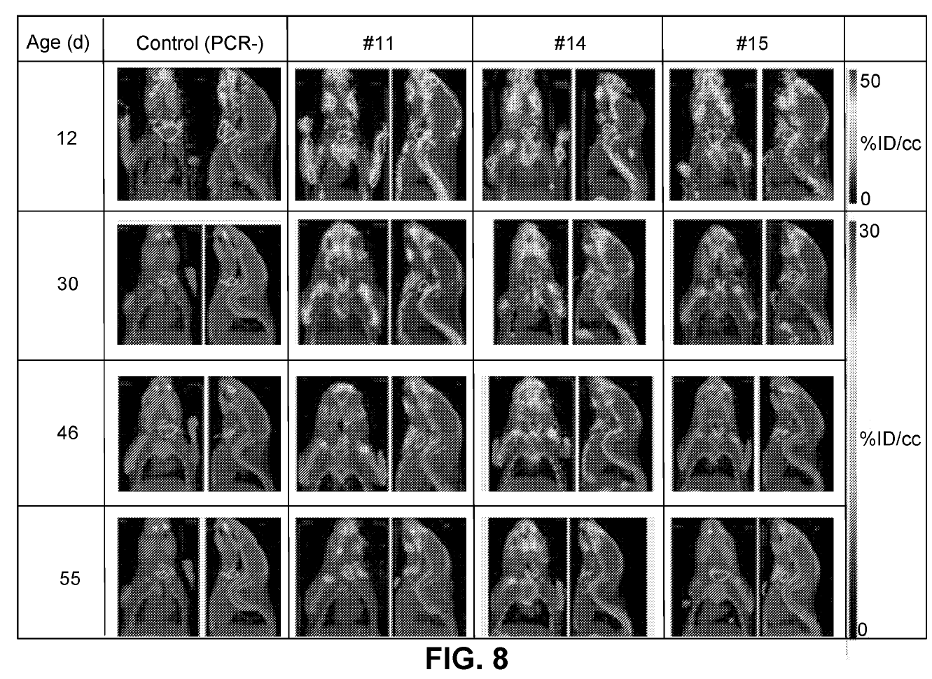

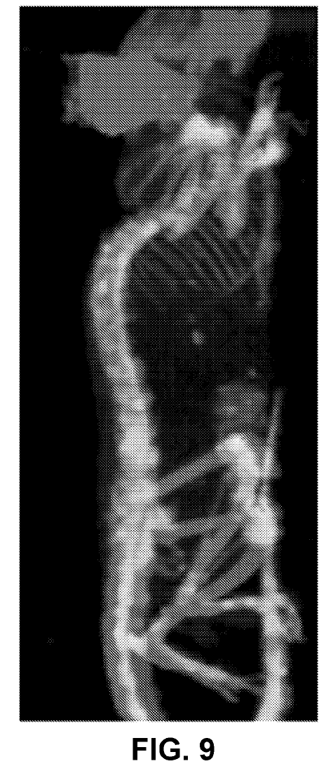

The rCol1α1pro/hNIS mice also were imaged early after birth and followed over time. From these results, NIS polypeptide expression was observed to change over time (FIGS. 8 and 9). In particular, the NIS signals were strong in the growth plates, vertebrate, and joints, but this diminished over time as the mice matured.

### Example 2—Following Rotator Cuff Injury in Transgenic NIS Animals

Rotator cuff injury was induced on the left shoulder of transgenic rCol1α1pro/hNIS rats. The right shoulder was used as a non-injury control. Rotator cuff injury also was induced on the left shoulder of non-transgenic control rats.

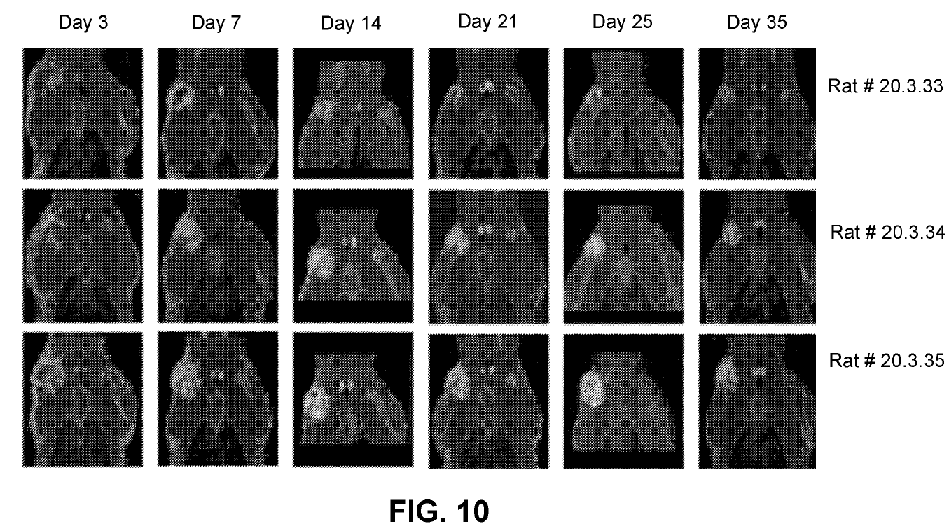

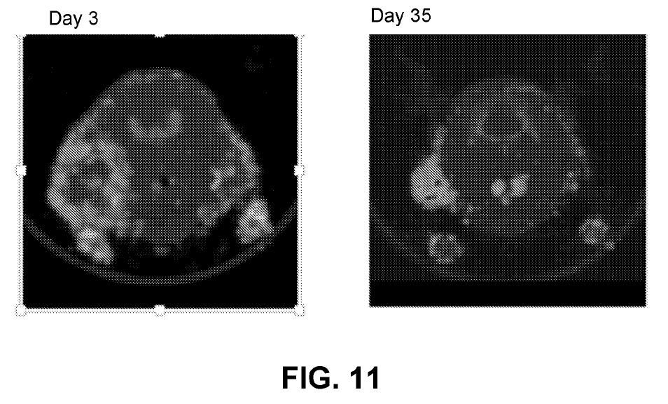

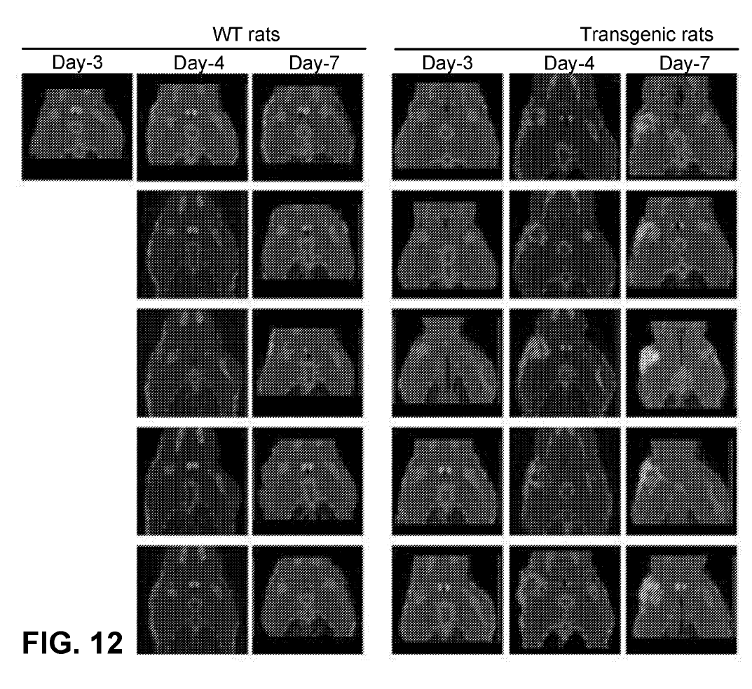

SPECT-CT imaging was performed to monitor the recruitment of cells expressing hNIS as a surrogate for type 1 collagen expression at days 3, 7, 14, 21, 25, and 35 days post injury (FIGS. 10 and 11). The baseline NIS expression in the skin (high collagen synthesis) remained stable, while there was increasing NIS expression in the shoulder/rotator cuff area for the rats undergoing active fibrogenesis post rotator cuff injury (FIGS. 10 and 11). It was apparent from the images that NIS imaging yielded important information on the variability of fibrogenesis in different rodents and allowed for an in-depth analysis into understand the correlation between fibrogenesis and the quality of repair in living animals over time. The control non-transgenic rats did not exhibit any change in uptake of radioactive material at baseline, before surgery (day −3), or post-surgery (FIG. 12). In contrast, the transgenic rCol1α1pro/hNIS rats exhibited a strong NIS expression circling the site of rotator cuff injury (FIG. 12).

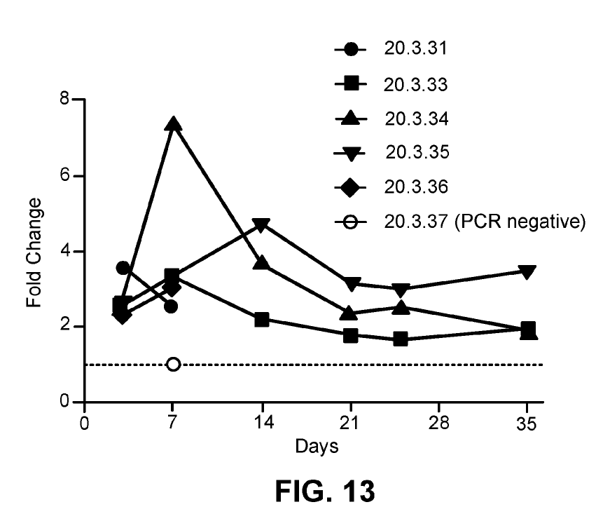

One hour before SPECT-CT imaging, rats were injected with Tc99m at 1 mCi/rat. NIS signals on both shoulders were quantitated using PMOD program. The fold increase of NIS uptake signal on the injured over control shoulder in each animal was determined (FIG. 13). Rat #20.3.37 was PCR negative and was used as negative control. There was no increase in NIS expression between both shoulders in the control rat.

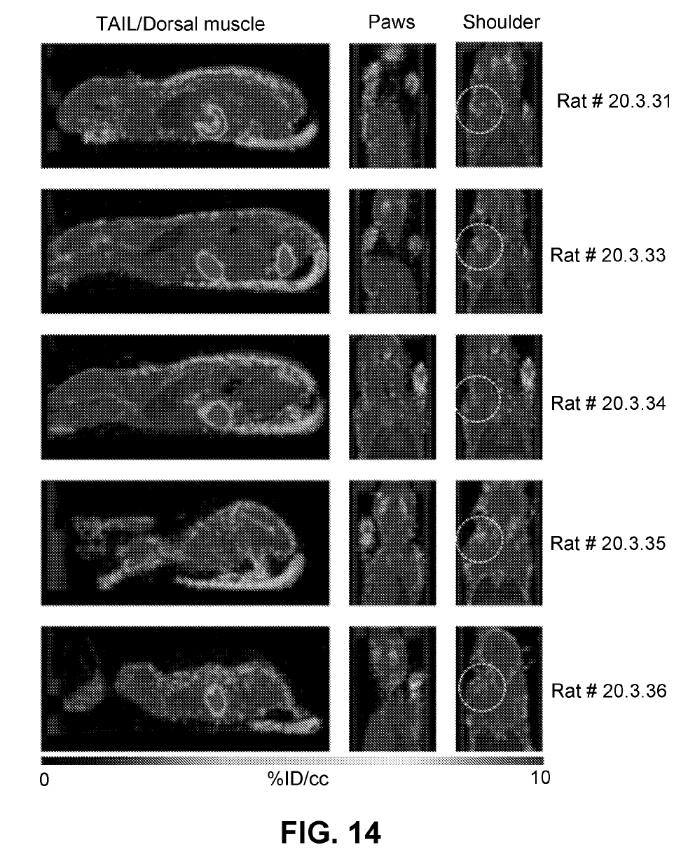

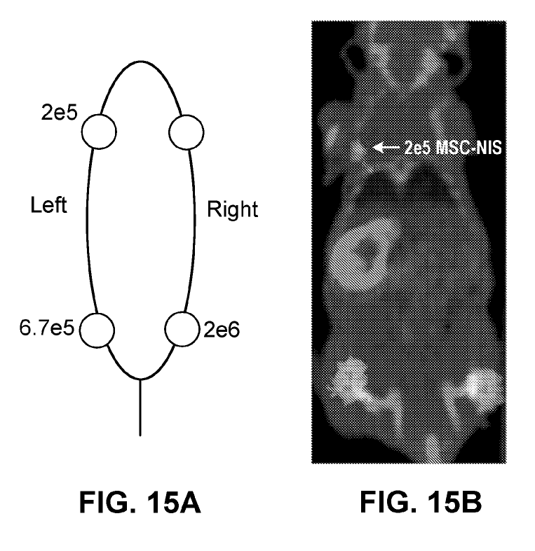

In addition, skin NIS expression in the transgenic rCol1α1pro/hNIS rats was stable over time. Stable NIS expression in the skin over time was observed in control Cola1-A1-NIS transgenic rats that did not receive rotator cuff injury (FIG. 14).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of monitoring a pathological process over time within a living transgenic non-human animal comprising somatic and germline cells comprising a promoter sequence operably linked to a nucleic acid sequence encoding a NIS polypeptide, wherein said method comprises:
(a) administering a first preparation comprising a radiotracer to said animal during a first time point, wherein said radiotracer is transported into a cell that expresses said NIS polypeptide,
(b) collecting imaging data of said radiotracer from within said animal to identify cells containing said radiotracer following said administration of said first preparation,
(c) administering a second preparation comprising a radiotracer to said animal during a second time point after said first time point, and
(d) collecting imaging data of said radiotracer from within said animal to identify cells containing said radiotracer following said administration of said second preparation.

2. The method of claim 1, wherein said pathological process is fibrosis.

3. The method of claim 1, wherein said animal is a mouse or rat.

4. The method of claim 1, wherein said promoter sequence is a Col1α1 promoter sequence.

5. The method of claim 1, wherein said NIS polypeptide is a human NIS polypeptide.

6. The method of claim 1, wherein said second time point is at least 5 days after said first time point.

7. The method of claim 1, wherein said second time point is at least 20 days after said first time point.

8. The method of claim 1, wherein said radiotracer is pertechnetate or radioiodide.

9. A transgenic non-human animal, whose somatic and germline cells comprise a transgene comprising a promoter sequence operably linked to a nucleic acid sequence encoding a NIS polypeptide, wherein radiotracer images of said animal at different time points, when a radiotracer that is transported into cells that express said NIS polypeptide is administered to said animal, provide imaging data with observable differences in the stages of a pathological process within said animal at said different time points.

10. The transgenic non-human animal of claim 9, wherein said pathological process is fibrosis.

11. The transgenic non-human animal of claim 9, wherein said animal is a mouse or rat.

12. The transgenic non-human animal of claim 9, wherein said promoter sequence is a Col1α1 promoter sequence.

13. The transgenic non-human animal of claim 9, wherein said NIS polypeptide is a human NIS polypeptide.

14. The transgenic non-human animal of claim 9, wherein said different time points are at least 5 days apart.

15. The transgenic non-human animal of claim 9, wherein said different time points are at least 20 days apart.

16. The transgenic non-human animal of claim 9, wherein said radiotracer is pertechnetate or radioiodide.

17. A transgenic non-human animal, whose somatic and germline cells comprise a transgene comprising a promoter sequence operably linked to a nucleic acid sequence encoding a NIS polypeptide, wherein said promoter sequence is a Col1α1 promoter sequence, an osteocalcin promoter sequence, a bone sialoprotein (BSP) promoter sequence, a VEGFR2 promoter sequence, a prostate-specific antigen (PSA) promoter sequence, an Epo promoter sequence, a serum amyloid A-1 (Saa1) promoter sequence, a TNF promoter sequence, an iNOS promoter sequence, a 3 NFkB site from the Igk light chain promoter sequence, an heme oxygenase-1 (Hol or Hmox-1) promoter sequence, a superoxide dismutase (SOD1) promoter sequence, a cytochrome p450 isoform 3A (Cyp3A4) promoter sequence, a rat insulin gene (RIP) promoter sequence, a resistin (Retn) promoter sequence, or an elastase I (EL1) promoter sequence.


SEQUENCE LISTING (6 sequences)

SEQ ID NO: 1 - DNA, 23 bp, Artificial Sequence
  synthetic oligonucleotide primer
gctctcctccctgctaacgactc

SEQ ID NO: 2 - DNA, 23 bp, Artificial Sequence
  synthetic oligonucleotide primer
agacgatcctcattggtgggcag

SEQ ID NO: 3 - DNA, 21 bp, Artificial Sequence
  synthetic oligonucleotide primer
tgcgtggctctctcagtcaac

SEQ ID NO: 4 - DNA, 21 bp, Artificial Sequence
  synthetic oligonucleotide primer
agttcttcaggacccttgacc

SEQ ID NO: 5 - DNA, 21 bp, Artificial Sequence
  synthetic oligonucleotide primer
tgcgtggctctctcagtcaac

SEQ ID NO: 6 - DNA, 20 bp, Artificial Sequence
  synthetic oligonucleotide primer
attggtgggcaggaagccag